(19) United States
(12) Patent Application Publication
(10) Pub. No.: US 2010/0228178 A1
(43) Pub. Date: Sep. 9, 2010

(54) HEMODIALYSIS CATHETER APPARATUS

(76) Inventor: J. Kevin McGraw, Dublin, OH (US)

Correspondence Address: STETINA BRUNDA GARRED & BRUCKER, 75 ENTERPRISE, SUITE 250, ALISO VIEJO, CA, 92656, US

(21) Appl. No.: 12/399,645

(22) Filed: Mar. 6, 2009

(51) Int. Cl. A61M 1/14

(52) U.S. Cl. 604/6.16

(57) ABSTRACT

A catheter apparatus is provided for reducing blood clotting in connection with hemodialysis treatment. The catheter apparatus can comprise a first conduit defining an arterial lumen and a second conduit defining a venous lumen. Large staggered apertures can be provided in side walls of the conduits with at least one of the apertures having a cross-sectional area equal or greater than a cross-sectional area of one of the lumens. The catheter apparatus can further include first and second removable obturators adapted for axial insertion into the conduits and for occluding the apertures while so inserted. Advantageously, the obturators can wipe the apertures during removal, causing blood clots incident on the apertures to be dislodged from the apertures. Related methods for using the catheter apparatus in performing hemodialysis treatment are also provided.

### CROSS-REFERENCE TO RELATED APPLICATIONS

This application claims the benefit of U.S. Provisional Application No. 60/693,299 entitled “HEMODIALYSIS CATHETER APPARATUS” filed Jun. 23, 2005, the entirety of the disclosure of which is expressly incorporated herein by reference.

### STATEMENT RE: FEDERALLY SPONSORED RESEARCH/DEVELOPMENT

Not Applicable

### BACKGROUND

The present disclosure relates generally to catheters, and more particularly to catheters for use in hemodialysis treatment.

In the area of health care, it is often necessary to perform hemodialysis treatment on patients that have malfunctioning or failed kidneys. Such procedures are typically performed on a periodic basis and can require up to several treatments per week.

Hemodialysis treatments can sometimes require the insertion of a catheter into a patient in order to perform the treatment. However, because of the frequency of such treatments, it can become inconvenient to remove the catheter after treatment and re-insert the catheter for the next treatment. This is especially true for procedures which require insertion of the catheter into the patient's groin area which can cause substantial patient discomfort. As a result, it is often preferable to leave the catheter in place between treatments.

One primary problem associated with leaving the catheter in place is the tendency for blood to clot at openings of the catheter, thereby obstructing blood flow through the catheter during subsequent uses. When blood flow is obstructed, the time required to perform hemodialysis can substantially increase due to the greater length of time required to process a given volume of the patient's blood. This can result in further inconvenience to the patient and increased health care expenses related to the operation of hemodialysis equipment for longer periods of time.

One approach to removing such blood clotting involves the introduction of Heparin into the catheter after each use. Unfortunately, the use of Heparin in conjunction with every successive dialysis treatment can complicate the administration of such treatments while simultaneously increasing costs.

To reduce the possibility of clotting, prior art catheters are often additionally limited in the number and size of apertures provided for receiving the patient's blood and returning processed blood back to the patient. Unfortunately, these limits on the apertures can also lead to prolonged dialysis time due to reduced blood flow rates.

Accordingly, there exists a need for an improved catheter apparatus and associated methods that reduce blood clotting and promote high blood flow rates in connection with hemodialysis treatment.

### BRIEF SUMMARY

The present disclosure, roughly described, provides an improved catheter apparatus and methods associated therewith for reducing blood clotting and improving blood flow in connection with hemodialysis treatment. The catheter apparatus can comprise a first conduit defining an arterial lumen, the first conduit having proximal and distal ends, and a second conduit defining a venous lumen, the second conduit having proximal and distal ends. The second conduit and venous lumen can be shaped to be longer than the first conduit and arterial lumen.

A plurality of staggered first apertures can be provided in a side wall of the first conduit for receiving blood therethrough into the arterial lumen during hemodialysis treatment, with at least one of the first apertures having a cross-sectional area equal or greater than a cross-sectional area of the arterial lumen. The first apertures may decrease in size upon approaching the distal end of the first conduit. A plurality of staggered second apertures can also be provided in a side wall of the second conduit for expelling blood therethrough from the venous lumen during hemodialysis treatment, with at least one of the second apertures having a cross-sectional area equal or greater than a cross-sectional area of the venous lumen. The second apertures may increase in size upon approaching the distal end of the second conduit. Furthermore, the first apertures may include at least two apertures of different sizes, and the second apertures may include at least two apertures of different sizes. Finally, it is also contemplated that the size of each of the first apertures may successively decrease upon approaching the distal end of the first conduit, and that the size of each of the second apertures may successively increase upon approaching the distal end of the second conduit.

The catheter apparatus can further include first and second removable obturators adapted for axial insertion into the proximal ends of the respective ones of the first and second conduits. In addition, the obturators can be fashioned to occlude the apertures while inserted into the conduits. Advantageously, the obturators can be adapted for wiping the apertures during removal of the obturators from the conduits. As a result of the wiping of the apertures by the obturators, blood clots incident on the apertures can be dislodged from the apertures.

The first obturator can comprise a first elongate sheath approximately equal in length to the first conduit and having a diameter approximately equal to a diameter of the arterial lumen. Similarly, the second obturator can comprise a second elongate sheath approximately equal in length to the second conduit and having a diameter approximately equal to a diameter of the venous lumen. The first obturator may lockably engage the proximal end of the first conduit while the first obturator is inserted into the first conduit, and the second obturator may lockably engage the proximal end of the second conduit while the second obturator is inserted into the second conduit.

At least a portion of the first and second conduits can be implemented to share a common wall and comprise a substantially cylindrical elongate body. In addition, each of the first and second conduits may define a substantially semi-circular cross-section.

In another embodiment, a method for performing hemodialysis treatment using a catheter apparatus is provided. A distal end of the catheter apparatus can be inserted into a patient, preferably into a vascular structure of the patient. First and second removable obturators can be axially inserted into proximal ends of the respective ones of first and second conduits, of the catheter apparatus. While inserted into the conduits, the first and second obturators occlude apertures in side walls of the catheter apparatus. The obturators can be removed from the conduits, resulting in the obturators wiping the apertures and causing blood clots incident on the apertures to be dislodged from the apertures.

Conduits of the catheter device can be connected to hemodialysis equipment. Thereafter, blood from the patient can be allowed to flow through an arterial lumen of the catheter apparatus to the hemodialysis equipment for treatment, and blood treated by the hemodialysis equipment can be allowed to flow through a venous lumen of the catheter apparatus to the vascular structure of the patient. Following hemodialysis treatment, third and fourth removable obturators can be axially inserted into the proximal ends of the respective ones of the first and second conduits, with the third and fourth obturators occluding the apertures while inserted into the conduits.

These as well as other embodiments contemplated by the present disclosure will be more fully set forth in the detailed description below and the figures submitted herewith.

### BRIEF DESCRIPTION OF THE DRAWINGS

FIG. 1 is a top view of a catheter apparatus for use in hemodialysis treatment in accordance with an embodiment of the present disclosure;

FIG. 2 is a top view of distal ends of arterial and venous conduits of a catheter apparatus in accordance with an embodiment of the present disclosure;

FIG. 3 is a cross-sectional view of arterial and venous conduits of a catheter apparatus in accordance with an embodiment of the present disclosure;

FIG. 4 is a side view of a distal end of a venous conduit exhibiting a plurality of staggered apertures in accordance with an embodiment of the present disclosure;

FIG. 5 is a side view of a distal end of an arterial conduit exhibiting a plurality of staggered apertures in accordance with an embodiment of the present disclosure;

FIG. 6 is a cross-sectional view of an obturator partially inserted into a conduit of a catheter apparatus in accordance with an embodiment of the present disclosure; and

FIG. 7 is a cross-sectional view of an obturator fully inserted into a conduit of a catheter apparatus in accordance with an embodiment of the present disclosure.

### DETAILED DESCRIPTION

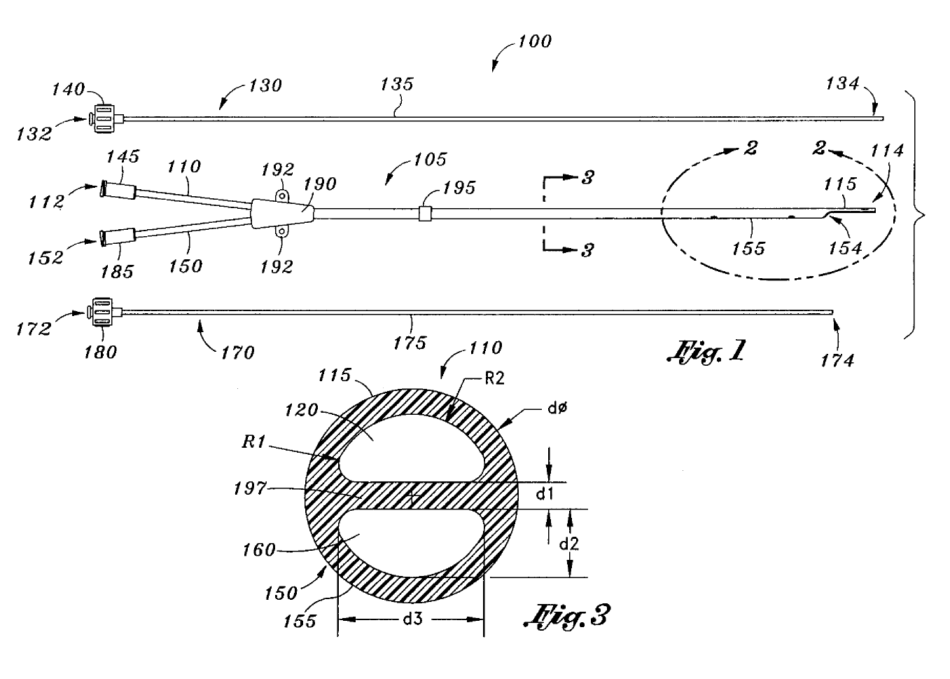

FIG. 1 is a top view of a catheter apparatus 100 for use in hemodialysis treatment in accordance with an embodiment of the present disclosure. As illustrated, the apparatus 100 can include a main body portion 105, a venous obturator 130, and an arterial obturator 170.

Turning first to the main body portion 105, an arterial conduit 150 is provided for receiving blood from a patient through one or more apertures at an open distal end 154 of the conduit 150, and passing the blood to dialysis equipment (not shown) through a proximal end 152 of the conduit 150. A venous conduit 110 is provided for receiving blood from the dialysis equipment through proximal end 112 of the conduit 110 and passing the blood back to the patient through one or more apertures at an open distal end 114 of the conduit 110. In one embodiment, conduits 10 and 150 can be implemented using transparent PVC material.

The proximal end 112 of venous conduit 110 can be provided with a venous connector 145 for connecting the conduit 110 to dialysis equipment and for receiving a venous obturator 130, as further described herein. Similarly, the proximal end 152 of arterial conduit 150 can be provided with an arterial connector 185 for connecting the conduit 150 to dialysis equipment and for receiving an arterial obturator 170, as further described herein. In one embodiment, venous connector 145 can be blue in color and arterial connector 185 can be red in color. As illustrated, the distal end 114 of conduit 110 extends beyond the distal end 154 of conduit 150 in order to reduce recirculation of blood processed by the dialysis equipment from the venous conduit 110 back into the arterial conduit 150.

Conduits 110 and 150 are physically separated into separate shafts at distal ends 112 and 152. As the conduits 110 and 150 pass through anchor 190, they are joined by a shared common wall and comprise a substantially cylindrical elongate body extending from anchor 190 to distal end 154. Anchor 192 also comprises a plurality of suturing tabs 192 useful for securing the main body portion 105 when in use. Main body portion 105 further comprises a cuff 195 encircling the body 105 for providing frictional resistance to prevent inadvertent removal of the body 105 from a patient.

FIG. 2 is a top view of distal ends 114 and 154 of venous and arterial conduits 110 and 150, respectively, of a catheter apparatus 100 taken at line 2-2 of FIG. 1. Venous conduit 110 and arterial conduit 150 include side walls 115 and 155, respectively, as well as shared wall 197. Side wall 115 and shared wall 197 of the venous conduit 110 define a venous lumen 120 for passing blood through the conduit 110. Similarly, side wall 155 and shared wall 197 of the arterial conduit 150 define an arterial lumen 160 for passing blood through the conduit 150. Apertures in side walls 115 and 155 of the conduits are also provided, as further described herein.

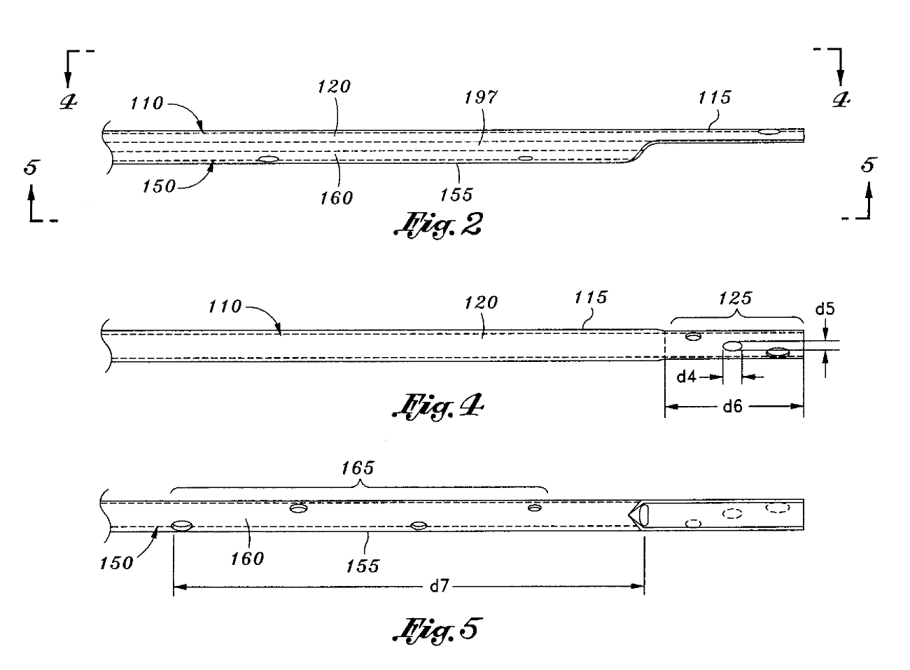

FIG. 3 is a cross-sectional view of catheter apparatus 100 taken at line 3-3 of FIG. 1. As illustrated, each of the conduits 110 and 150 and lumens 120 and 160 exhibit substantially semi-circular cross-sections. In one embodiment, the dimensions d0 (outside diameter of body 105), d1 (thickness of shared wall 197), d2 (first lumen dimension), d3 (second lumen dimension), R1 (first radius), and R2 (second radius) can be implemented as 4.83 mm, 0.64 mm, 1.46 mm, 3.18 mm, 0.38 mm, and 1.78 mm, respectively. In addition, each of lumens 120 and 160 can be implemented having a cross-sectional area of 3.69 mm². However, it will be appreciated that such dimensions can vary in other embodiments.

FIG. 4 is a side view of a distal end of a venous conduit 110 taken at line 4-4 of FIG. 2. A plurality of apertures 125 (i.e. fenestrations) are provided in side wall 115 of conduit 110. The apertures 125 serve to expel blood from the venous lumen 120 back to the patient during hemodialysis treatment.

As illustrated, the apertures 125 can be offset from each other in a staggered orientation in order to reduce the likelihood of blockage of the apertures 125 during hemodialysis treatment. For example, if the side wall 115 of the catheter apparatus 100 is positioned against an internal wall of a patient's vascular structure, such as an artery or vein, the staggered orientation can prevent at least some of the apertures 125 from being blocked by the internal wall. Although the apertures 125 are illustrated as being elliptical, it will be appreciated that alternative shapes can be used for the apertures 125 including but not limited to non-elliptical or circular shapes. In addition, although three apertures 125 are illustrated in FIG. 4, it will be appreciated that catheter apparatus 100 can be implemented with any number of apertures 125.

In various embodiments, some or all of the apertures 125 can be sized to exhibit a cross-sectional area equal or greater than a cross-sectional area of the venous lumen 120. Such sizing can significantly improve blood flow over prior catheters having smaller apertures, Moreover, blood clotting incident to the apertures 125 can be substantially eliminated through operation of obturator 130 as further described herein. In one embodiment, dimensions d4, d5, and d6 of the apertures 125 can be implemented as 3.05 mm, 1.53 mm, and 25.4 mm, respectively, with each aperture 125 having a cross-sectional area of 3.69 mm². However, it will be appreciated that such dimensions can vary in other embodiments.

Additionally, it is also contemplated that the size of apertures 125 can be implemented with the same or different sizes, such as with the size of each aperture 125 being larger in size than an aperture 125 immediately to its left, resulting in increasing sizes for apertures 125 closer to the distal end 114 of venous conduit 110. Such a configuration, in which the size of apertures 125 decreases toward the distal end 114 of venous conduit 110, recirculation of blood processed by the dialysis equipment from the venous conduit 110 back into the arterial conduit 150.

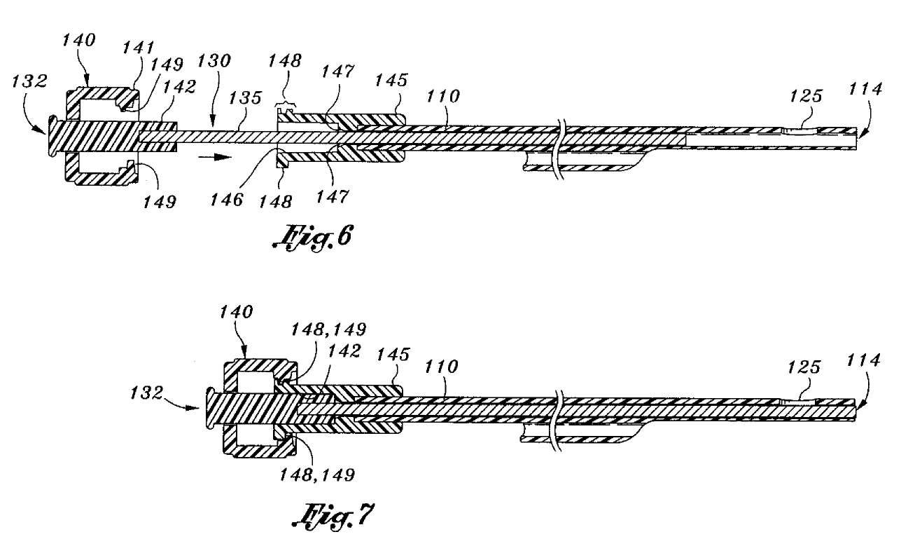

FIG. 5 is a side view of a distal end 154 of an arterial conduit 150 taken at line 5-5 of FIG. 2. A plurality of apertures 165 (i.e. fenestrations) are provided in side wall 155 of conduit 150. The apertures 165 serve to receive blood into the arterial lumen 160 from the patient during hemodialysis treatment.

Similar to the discussion above with respect to FIG. 4, the apertures 165 of FIG. 5 can also be offset from each other in a staggered orientation in order to reduce the likelihood of blockage of the apertures 165 during hemodialysis treatment. Although the apertures 165 are illustrated as being elliptical, it will be appreciated that alternative shapes can be used for the apertures 165 including but not limited to non-elliptical or circular shapes. In addition, although four apertures 165 are illustrated in FIG. 53 it will be appreciated that catheter apparatus 100 can be implemented with any number of apertures 165.

In various embodiments, some or all of the apertures 165 can be sized to define a cross-sectional area equal or greater than a cross-sectional area of the arterial lumen 160. Such sizing can significantly improve blood flow over prior catheters having smaller apertures. Moreover, blood clotting incident to the apertures 165 can be substantially eliminated through operation of obturator 170 as further described herein.

As discussed above with respect to the size of apertures 125, other embodiments may be implemented with the size of apertures 165 being of the same or different sizes, such as with the size of each aperture 165 being smaller in size than an aperture 165 immediately to its left, resulting in decreasing sizes for apertures 165 closer to the distal end 154 of conduit 150. Alternatively, the size of apertures 165 can also be implemented with different sizes, with the size of each aperture 165 being smaller in size than an aperture 165 immediately to its right. However, it is contemplated that a configuration in which the size of apertures 165 decreases toward the distal end 154 of conduit 150 may tend to reduce recirculation of blood processed by the dialysis equipment from the venous conduit 10 back into the arterial conduit 150.

Furthermore, with respect to the apertures 1251165, the apertures 125/165 may each include a plurality of apertures, as shown in FIGS. 4 and 5. For example, as shown in FIG. 4, apertures 125 may include at least two apertures of different sizes. Further, as shown in FIG. 5, the apertures 165 may also include at least two apertures of different sizes. Furthermore, the size of each of the apertures 125 may successively increase upon approaching the distal end 114 of the conduit 110, and the size of each of the apertures 165 may successively increase upon approaching the distal end 154 of the conduit 150. Thus, if three or more apertures 125/165 are used, each aperture 125/165 may have a different size than the other respective apertures 125/165, such as to allow the apertures 125/165 to respectively increase and decrease in size with each successive aperture 125/165. Other such configurations and modifications may be implemented using the teachings herein in order to reduce recirculation of blood processed by the dialysis equipment from the venous conduit 110 back into the arterial conduit 150.

Therefore, the recirculation of blood processed by the dialysis equipment may tend to be further reduced when: 1) the size of each aperture 125 is larger in size than an aperture 125 immediately to its left, resulting in increasing sizes for apertures 125 closer to the distal end 114 of venous conduit 110; and 2) the size of each aperture 165 is smaller in size than an aperture 165 immediately to its left, resulting in decreasing sizes for apertures 165 closer to the distal end 154 of conduit 150.

As illustrated, apertures 165 can be distributed along a portion of side wall 155 denoted by dimension d7. In one embodiment, dimension d7 can be implemented as 10 cm. However, it will be appreciated that such dimension can vary in other embodiments.

Returning to FIG. 1, as discussed, catheter apparatus 100 can comprise a pair of obturators 130 and 170. Distal ends 134 and 174 of the obturators can be axially inserted into the proximal ends 112 and 152 of respective ones of conduits 110 and 150.

Obturators 130 and 170 each comprise flexible elongate sheathes 135 and 175, respectively, each having substantially semi-circular cross-sections. Sheathes 135 and 175 can be sized so as to completely fill lumens 120 and 160 and occlude apertures 125 and 165 while inserted into conduits 110 and 150. Caps 140 and 180 attached to sheathes 135 and 175 can be provided at the proximal ends 132 and 172 of the obturators for securing the obturators 130 and 170 to connectors 145 and 185 of conduits 110 and 150 while the obturators 130 and 170 are inserted.

The insertion of obturator 130 into conduit 110 can be further understood with reference to FIGS. 6 and 7. FIGS. 6 and 7 provide cross-sectional views of an obturator 130 partially (FIG. 6) and fully (FIG. 7) inserted into a conduit 110 of a catheter apparatus 100 in accordance with an embodiment of the present disclosure.

Referring to FIG. 6, obturator 130 comprises an elongate sheath 135 attached to a cap 140 at the proximal end 132 of the obturator 130. As illustrated, cap 140 comprises a ring portion 141 having a plurality of locking members 149. The ring portion 141 is connected to a male luer tapered portion 142.

Venous connector 145 comprises a plurality of locking members 148 and a female luer portion 146. As illustrated, the female luer portion 146 can receive the male luer portion 141 of cap 140. Venous connector 145 further comprises recessed lead-in portions 147 for facilitating the receiving of the proximal end 134 of obturator 130 into conduit 110.

Referring now to FIG. 7, obturator 130 is illustrated as being fully inserted into conduit 110. While inserted, the male luer portion 142 of cap 140 completely fills the female luer portion 146 of venous connector 145, with locking members 148 and 149 engaging each other.

In addition, as illustrated at distal end 114, the sheath 135 of obturator 130 occludes apertures 125 while inserted into the conduit 110. Accordingly, it will be appreciated that obturator 130 can be inserted into conduit 110 after hemodialysis treatment has been completed and while the main body 105 of the catheter apparatus 100 remains inserted in a patient. Due to the occlusion of the apertures 125 by the obturator 130, blood clotting at the apertures 125 can be substantially eliminated. When it is desired to perform dialysis treatment, obturator 130 can be removed from conduit 110. Upon such removal, a wiping action between the obturator 130 and apertures 125 additionally removes any minor clotting incident at the apertures 125. Furthermore, subsequent flow of blood through the apertures 125 may also tend to remove any minor clotting incident at apertures 125.

It will be appreciated that the insertion and removal of obturator 170 with respect to conduit 150 can be performed in substantially the same manner as described above with respect to obturator 130 and conduit 110. As such, obturator 170 can occlude apertures 165 and also perform a wiping action therewith to remove clotting incident at the apertures 165. Thus, the novel features discussed above with respect to obturator 130 and conduit 110 may also apply for the obturator 170 and conduit 150.

As discussed, catheter apparatus 100 is adapted for use in hemodialysis treatment. In operation, a substantial portion of the main body 105 of the apparatus can be initially inserted into a patient. In particular, the distal ends 114 and 154 of conduits 110 and 150 may be inserted into a patient's vascular structure. If it is desired that hemodialysis treatment not be performed immediately, then obturators 130 and 170 can be inserted into respective ones of conduits 110 and 150, and secured to connectors 145 and 185 through locking members 148 and 149. The presence of the obturators in the conduits 110/150 prevents the accumulation of blood clots within the lumens 120/150 as well as on apertures 125/165 and open distal ends 114/154 of the conduits 110/150.

When it is desired to perform hemodialysis treatment, then obturators 130/170 can be removed from conduits 110/150, causing a wiping action between the obturators 130/170 and apertures 125/165 to remove clotting incident at the apertures 125/165. Appropriate hemodialysis equipment can then be connected to connectors 145 and 185, thereby providing a fluid path for the patient's blood to pass through lumens 120 and 150 of the conduits. During hemodialysis treatment, the sizing and placement of apertures 125/165, as well as the removal of blood clots incident thereto, allow for a high blood flow rate through the catheter apparatus 100.

With particular regard to the wiping action between the obturators 130/170 and apertures 125/165, the obturators may be formed to include a variable surface texture that enhances the wiping action. For example, the surface texture may be configured to include raised hair-like bristles. Thus, as the obturators 130/170 are removed, the bristles may contact the apertures 125/165 and jostle loose any clotting incident at the apertures 125/165. The surface texture may be formed in a variety of manufacturing processes and is not limited to bristles, but may include other shapes and types of surface textures. In particular, the surface texture should be designed and configured to provide sufficient frictional contact with the apertures 125/165 in order to provide proper removal of clotting. Further, the surface texture may be formed continuously along the length of the obturators 130/170 or selectively along the lengths thereof, such as one or more rings, a helical pattern, etc. Thus, upon removing the obturators 130/170, the surface texture of the obturators 130/170 may further facilitate wiping action between the obturators 130/170 and the apertures 125/165 to remove clotting incident at the apertures 125/165. In addition to removing the obturators 130/170 with a single extraction stroke, the obturators 125/165 may be selectively moved axially back and forth or rotated past the apertures 125/165 in order to enhance the removal of clotting from the apertures 125/165.

After hemodialysis treatment is finished, a new set of sterile obturators 130/170 can be inserted into the respective ones of the conduits 110 and 150 and secured to connectors 145 and 185 through locking members 148 and 149. In this regard, it will be appreciated that obturators 130/170 can be implemented as sterile, disposable items that are replaced after each hemodialysis treatment. While inserted, the presence of the obturators 130/170 in the conduits 110/150 prevents the accumulation of blood clots within the lumens 120/150 as well as on apertures 125/165 and open distal ends 114/154 of the conduits 110/150 between successive hemodialysis treatments. When it is desired to commence hemodialysis treatment again, the new obturators 130/170 can be removed in the manner previously discussed.

It will be appreciated that various aspects of the present disclosure provide significant advantages over prior approaches to hemodialysis catheters. The use of obturators 130/170 to reduce blood clotting removes the need for costly repetitive applications of Heparin to the catheter apparatus 100 before or after hemodialysis treatment. In addition, the reduced blood clotting at the apertures 125/165 allows for the apertures to be sized with large cross-sectional areas, thereby improving blood flow rates through the apparatus 100.

The foregoing disclosure is not intended to limit the present disclosure to the precise forms or particular fields of use disclosed. It is contemplated that various alternate embodiments and/or modifications to the present disclosure, whether explicitly described or implied herein, are possible in light of the disclosure. Where applicable, the ordering of various steps described herein can be changed, combined into composite steps, and/or dissected into sub-steps to provide features described herein.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. A catheter apparatus for use in hemodialysis treatment, the catheter apparatus comprising:
a first conduit defining an arterial lumen and an arterial length, the first conduit having proximal and distal ends, the arterial lumen having a first cross sectional configuration that extends along a substantial portion of the arterial length of the arterial lumen;
a second conduit defining a venous lumen and a venous length, the second conduit having proximal and distal ends;
a plurality of first apertures in a side wall of the first conduit for receiving blood therethrough into the arterial lumen during hemodialysis treatment;
a plurality of second apertures in a side wall of the second conduit for expelling blood therethrough from the venous lumen during hemodialysis treatment; and
a first removable obturator adapted for axial insertion in the proximal end of the first conduit, the first obturator occluding the plurality of first apertures while inserted into the first conduit, at least a portion of the first obturator includes a textured surface for dislodging blood clots incident on the plurality of first apertures or in the arterial lumen.

22. The apparatus of claim 21 wherein the first and second cross sectional configurations are semi circular.

23. The apparatus of claim 21 wherein the venous lumen has a second cross sectional configuration along a substantial portion of the venous length, and further including a second removable obturator adapted for axial insertion into the proximal end of the second conduit, the second obturator occluding the plurality of second apertures while inserted into the second conduit, at least a portion of the second obturator includes a textured surface for dislodging blood clots incident on the plurality of second apertures or in the venous lumen.

24. The apparatus of claim 21 wherein at least a portion of the first and second conduits share a common wall.

25. The apparatus of claim 21 wherein the plurality of first and second apertures are substantially elliptical.

26. The apparatus of claim 21 wherein the plurality of first and second apertures are substantially circular.

27. The apparatus of claim 21 wherein at least one first aperture of the plurality of first apertures defines a cross-sectional area equal or greater than the cross-sectional area of the first conduit, and at least one second aperture of the plurality of second apertures defines a cross-sectional area equal or greater than the cross-sectional area of the second conduit.

28. The apparatus of claim 21 wherein the plurality of first apertures are staggered in the side wall of the first conduit, and the plurality of second apertures are staggered in the side wall of the second conduit.

29. The apparatus of claim 21 wherein at least a portion of the first and second conduits collectively comprise an elongate body, the apparatus further including a cuff circumscribing the elongate body and providing frictional resistance to prevent inadvertent removal of the catheter apparatus from a patient.

30. The apparatus of claim 21 wherein the venous length is longer than the arterial length.

31. The apparatus of claim 23 wherein the textured surfaces of the first and second obtruators include a plurality of bristles at distal end portions thereof for wiping the plurality of first and second apertures during removal of the obturators from the first and second conduits to dislodge blood clots incident on the plurality of first and second apertures.

32. The apparatus of claim 31 further wherein the first obturator further comprises a first elongate sheath approximately equal in length to the first conduit and having a diameter approximately equal to a diameter of the arterial lumen, and the second obturator further comprises a second elongate sheath approximately equal in length to the second conduit and having a diameter approximately equal to a diameter of the venous lumen.

33. The apparatus of claim 32 wherein the first obturator is configured to lockably engage the proximal end of the first conduit while the first obturator is inserted into the first conduit, and the second obturator is configured to lockably engage the proximal end of the second conduit while the second obturator is inserted into the second conduit.

34. A catheter apparatus for use in hemodialysis treatment, the catheter apparatus comprising:
a first conduit defining an arterial lumen, the first conduit having proximal and distal ends;
a second conduit defining a venous lumen, the second conduit having proximal and distal ends;
at least three successive first apertures in a side wall of the first conduit for receiving blood therethrough into the arterial lumen during hemodialysis treatment, each successive aperture of the at least three successive first apertures successively decreasing in cross sectional area in a direction toward the distal end of the first conduit; and
a plurality of second apertures in a side wall of the second conduit for expelling blood therethrough from the venous lumen during hemodialysis treatment.

35. The apparatus of claim 34 wherein at least two of the at least three successive second apertures have different sizes.

36. The apparatus of claim 34 wherein the plurality of second apertures comprises at least three successive second apertures successively increasing in cross sectional area in a direction toward the distal end of the second conduit.
